(19) United States
(12) Patent Application Publication
(10) Pub. No.: US 2004/0192737 A1
(43) Pub. Date: Sep. 30, 2004

(54) Use of thiazolidinediones for the treatment of hyperglycaemia

(75) Inventors: Robin Edwin Buckingham, Welwyn Garden City (GB); Stephen Alistair Smith, Bramfield (GB)

Correspondence Address: GLAXOSMITHKLINE, Corporate Intellectual Property - UW2220, P.O. Box 1539, King of Prussia, PA, 19406-0939, US

(73) Assignee: SmithKline Beecham p.l.c.

(21) Appl. No.: 10/822,978

(22) Filed: Apr. 13, 2004

Related U.S. Application Data
(63) Continuation of application No. 10/154,725, filed on May 24, 2002.
Application No. 10/154,725 is a continuation of application No. 09/949,585, filed on Sep. 10, 2001, now abandoned.
Application No. 09/949,585 is a continuation of application No. 09/529,527, filed on Apr. 13, 2000, now abandoned.
Application No. 09/529,527 is a 371 of international application No. PCT/GB98/03067, filed on Oct. 12, 1998.

(30) Foreign Application Priority Data
Oct. 13, 1997 (GB) GB 9721692.3

(51) Int. Cl. A61K 31/4439; A61K 31/426

(52) U.S. Cl. 514/342; 514/369

(57) ABSTRACT

A method for the treatment of hyperglycaemia wherein plasma glucose levels in the range of from >126 mg/dl to 140 mg/dl, which method comprises administering an effective nontoxic and pharmaceutically acceptable amount of an insulin sensitiser, to a mammal in need thereof.

This invention relates to a method of treatment, in particular to a method for the treatment of a certain, specified hyperglycaemia.

European Patent Application, Publication Number 0,306,228 relates to certain thiazolidinedione derivatives disclosed as having hypoglycaemic and hypolipidaemic activity. One particular thiazolidinedione disclosed in EP 0306228 is 5-[4-[2-(N-methyl-N-(2-pyridyl)amino)ethoxy]benzyl]thiazolidine-2,4-dione (hereinafter ‘Compound (I)’). WO94/05659 discloses certain salts of Compound (I) including the maleate salt.

Compound (I) is an example of a class of anti-hyperglycaemic agents known as ‘insulin sensitisers’. In particular Compound (I) is a thiazolidinedione insulin sensitiser.

European Patent Applications, Publication Numbers: 0008203, 0139421, 0032128, 0428312, 0489663, 0155845, 0257781, 0208420, 0177353, 0319189, 0332331, 0332332, 0528734, 0508740; International Patent Application, Publication Numbers 92/18501, 93/02079, 93/22445 and U.S. Pat. Nos. 5,104,888 and 5,478,852, also disclose certain thiazolidinedione insulin sensitisers.

Another series of compounds generally recognised as having insulin sensitiser activity are those typified by the compounds disclosed in International Patent Applications, Publication Numbers WO93/21166 and WO94/01420. These compounds are herein referred to as ‘acyclic insulin sensitisers’. Other examples of acyclic insulin sensitisers are those disclosed in U.S. Pat. No. 5,232,945 and International Patent Applications, Publication Numbers WO92/03425 and WO91/19702.

Examples of other insulin sensitisers are those disclosed in European Patent Application, Publication Number 0533933, Japanese Patent Application Publication Number 05271204 and U.S. Pat. No. 5,264,451.

The Report of the Expert Committee of the Diagnosis and Classification of Diabetes Mellitus (Diabetes Care, vol 20(7), 1997, 1183-1197) states that Type 2 diabetes is characterised by fasting plasma glucose levels of >126 mg/dl (where fasting is defined as no calorific intake for at least 8 hours). It is also described therein how the development of diabetes commonly occurs over a period of several years characterised by a rise in fasting serum glycaemia levels from levels generally considered to be normal—plasma glucose levels of approximately 110 mg/dl—through to the stated hyperglycaemia characteristic of frank Type 2 diabetes. The Report also refers to metabolic stages intermediate between normal glucose homeostasis and diabetes, including impaired glucose tolerance and impaired fasting glucose.

It is known from EP0306228 that Compound I is useful in the prophylaxis of hyperglycaemia and hence for the treatment of impaired glucose tolerance. International Patent Application, Publication number WO 95/07694 also discloses that thiazolidinediones can be used to treat impaired glucose tolerance to prevent or delay the onset of Type 2 diabetes mellitus. However, EP0306228 and WO 95/07694 do not disclose the treatment of any particular range of glycaemias.

It is now surprisingly indicated that Compound (I) provides a particularly beneficial effect on glycaemic control in the range of hyperglycaemia from >126 mg/dl to 140 mg/dl, thereby delaying or preventing further elevation of the hypergylcaemia.

Accordingly, the invention provides a method for the treatment of hyperglycaemia, especially fasting hyperglycaemia, wherein plasma glucose levels are in the range of from >126 mg/dl to 140 mg/dl, which method comprises administering an effective non-toxic and pharmaceutically acceptable amount of an insulin sensitiser, to a mammal in need thereof.

In a further aspect the invention provides a method for improving glycaemic control in conditions characterised by hyperglycaemia, especially fasting hyperglycaemias, wherein the improvement is provided wherein plasma glucose levels are in the range of from >126 mg/dl to 140 mg/dl, thereby delaying or preventing further elevation of the hypergylcaemia, which method comprises administering an effective non-toxic and pharmaceutically acceptable amount of an insulin sensitiser, to a mammal in need thereof.

In yet a further aspect, the invention provides a method for the prophylaxis of hyperglycaemia, especially fasting hyperglycaemia, wherein plasma glucose levels are >140 mg/dl, which method comprises administering an effective non-toxic and pharmaceutically acceptable amount of an insulin sensitiser, to a mammal in need thereof.

One particular group of conditions defined herein, in addition to being characterised by hyperglycaemia wherein fasting plasma glucose levels are in the range of from >126 mg/dl to 140 mg/dl are further characterised by hyperglycaemia wherein plasma glucose levels following an oral glucose tolerance test are <140 mg/dl.

A further group of conditions defined herein are those wherein in addition to being characterised by hyperglycaemia wherein fasting plasma glucose levels are in the range of from >126 mg/dl to 140 mg/dl, are further characterised by hyperglycaemias wherein plasma glucose levels following an oral glucose tolerance test are in the range of from 140 to <200 mg/dl.

Suitably the hyperglycaemia is that associated with the Type 2 diabetes mellitus syndrome.

A suitable insulin sensitiser is a thiazolidinedione insulin sensitiser.

A suitable thiazolidinedione insulin sensitiser is Compound (I).

Other suitable thiazolidinedione insulin sensitisers include (+)-5-[[4-[(3,4-dihydro-6-hydroxy-2,5,7,8-tetramethyl-2H-1-benzopyran-2-yl)methoxy]phenyl]methyl]-2,4-thiazolidinedione (or troglitazone), 5-[4-[(1-methylcyclohexyl)methoxy]benzyl]thiazolidine-2,4-dione (or ciglitazone), 5-[4-[2-(5-ethylpyridin-2-yl)ethoxy]benzyl]thiazolidine-2,4-dione (or pioglitazone) or 5-[(2-benzyl-2,3-dihydrobenzopyran)-5-ylmethyl)thiazolidine-2,4-dione (or englitazone)

In one particular aspect, the method comprises the administration of 2 to 12 mg of Compound (I), especially when administered per day.

Particularly, the method comprises the administration of 2 to 4, 4 to 8 or 8 to 12 mg of Compound (I) per day.

Particularly, the method comprises the administration of 2 to 4 mg of Compound (I), especially when administered per day.

Particularly, the method comprises the administration of 4 to 8 mg, such as greater than 4 for example 4.1, to 8 mg, of Compound (I), especially when administered per day.

Particularly, the method comprises the administration of 8 to 12 mg of Compound (I), especially when administered per day.

Preferably, the method comprises the administration of 2 mg of Compound (I), especially when administered per day.

Preferably, the method comprises the administration of 4 mg of Compound (I), especially when administered per day.

Preferably, the method comprises the administration of 8 mg of Compound (I), especially when administered per day.

It will be understood that the insulin sensitiser, such as compound (I) is administered in a pharmaceutically acceptable form, including pharmaceutically acceptable derivatives such as pharmaceutically acceptable salts, esters and solvates thereof, as appropriate.

Suitable pharmaceutically acceptable salted forms of the insulin sensitisers, such as Compound (I), include those described in the above mentioned patents and patent applications such as in EP 0306228 and WO94/05659 for Compound (I).

A preferred pharmaceutically acceptable salt for Compound (I) is a maleate.

Suitable pharmaceutically acceptable solvated forms of the insulin sensitisers, such as Compound (I), include those described in the above mentioned patents and patent applications, such as in EP 0306228 and WO94/05659 for Compound (I), in particular hydrates.

The thiazolidinedione insulin sensitisers, such as Compound (I), may exist in one of several tautomeric forms, all of which are encompassed herein either as individual tautomeric forms or as mixtures thereof. Certain of the insulin sensitisers, such as Compound (I), contain one or more chiral carbon atom, and hence can exist in two or more stereoisomeric forms: All such forms are encompassed herein whether as individual isomers or as mixtures of isomers, including racemates.

As used herein the term ‘pharmaceutically acceptable’ embraces both human and veterinary use: for example the term ‘pharmaceutically acceptable’ embraces a veterinarily acceptable compound.

As used herein the oral glucose tolerance test is that referenced in Diabetes Care 1997, vol 20(7), 1183-1197.

As used herein ‘elevated normal’ hyperglycaemia is to be taken as generally understood in the art, with reference for example to the Report of the Expert Committee of the Diagnosis and Classification of Diabetes Mellitus but is usually taken to mean glycaemias wherein plasma glucose levels are >110 mg/dl.

Suitably, plasma glucose levels are fasting plasma glucose levels.

In the method of the invention, the active medicaments are preferably administered in pharmaceutical composition form. As indicated above, such compositions can include both medicaments or one only of the medicaments.

Such compositions may be prepared by admixing an insulin sensitiser, such as Compound (I) and especially 2 to 12 mg thereof, and a pharmaceutically acceptable carrier therefor.

Usually the compositions are adapted for oral administration. However, they may be adapted for other modes of administration, for example parenteral administration, sublingual or transdermal administration.

The compositions may be in the form of tablets, capsules, powders, granules, lozenges, suppositories, reconstitutable powders, or liquid preparations, such as oral or sterile parenteral solutions or suspensions.

In order to obtain consistency of administration it is preferred that a composition of the invention is in the form of a unit dose.

Unit dose presentation forms for oral administration may be tablets and capsules and may contain conventional excipients such as binding agents, for example syrup, acacia, gelatin, sorbitol, tragacanth, or polyvinylpyrrolidone; fillers, for example lactose, sugar, maize-starch, calcium phosphate, sorbitol or glycine; tabletting lubricants, for example magnesium stearate; disintegrants, for example starch, polyvinylpyrrolidone, sodium starch glycollate or microcrystalline cellulose; or pharmaceutically acceptable wetting agents such as sodium lauryl sulphate.

The compositions are preferably in a unit dosage form in an amount appropriate for the relevant daily dosage.

Suitable dosages for the insulin sensitisers include those disclosed in the above mentioned patents and patent applications.

Suitable dosages, including unit dosages, of Compound (I) comprise 1, 2, 3, 4, 5, 6, 7, 8, 9, 10, 11 or 12 mg of Compound (I).

In the treatment the medicaments may be administered from 1 to 6 times a day, but most preferably 1 or 2 times per day.

In the treatment involving compounds other than Compound (I), the required dosages and formulations are generally as described in the above mentioned patent publications which as stated above are incorporated by reference herein: An example includes the administration of 200-800 mg of Troglitazone, for example 200, 300 or 400 mg.

The solid oral compositions may be prepared by conventional methods of blending, filling or tabletting. Repeated blending operations may be used to distribute the active agent throughout those compositions employing large quantities of fillers. Such operations are of course conventional in the art. The tablets may be coated according to methods well known in normal pharmaceutical practice, in particular with an enteric coating.

Oral liquid preparations may be in the form of, for example, emulsions, syrups, or elixirs, or may be presented as a dry product for reconstitution with water or other suitable vehicle before use. Such liquid preparations may contain conventional additives such as suspending agents, for example sorbitol, syrup, methyl cellulose, gelatin, hydroxyethylcellulose, carboxymethylcellulose, aluminium stearate gel, hydrogenated edible fats; emulsifying agents, for example lecithin, sorbitan monooleate, or acacia; non-aqueous vehicles (which may include edible oils), for example almond oil, fractionated coconut oil, oily esters such as esters of glycerine, propylene glycol, or ethyl alcohol; preservatives, for example methyl or propyl p-hydroxybenzoate or sorbic acid; and if desired conventional flavouring or colouring agents.

For parenteral administration, fluid unit dosage forms are prepared utilizing the compound and a sterile vehicle, and, depending on the concentration used, can be either suspended or dissolved in the vehicle. In preparing solutions the compound can be dissolved in water for injection and filter sterilized before filling into a suitable vial or ampoule and sealing. Advantageously, adjuvants such as a local anaesthetic, a preservative and buffering agents can be dissolved in the vehicle. To enhance the stability, the composition can be frozen after filling into the vial and the water removed under vacuum. Parenteral suspensions are prepared in substantially the same manner, except that the medicament is suspended in the vehicle instead of being dissolved, and sterilization cannot be accomplished by filtration. The compound can be sterilized by exposure to ethylene oxide before suspending in the sterile vehicle. Advantageously, a surfactant or wetting agent is included in the composition to facilitate uniform distribution of the compound.

Compositions may contain from 0.1% to 99% by weight, preferably from 10-60% by weight, of the active material, depending upon the method of administration.

The composition may, if desired, be in the form of a pack accompanied by written or printed instructions for use.

The compositions are prepared and formulated according to conventional methods, such as those disclosed in standard reference texts, for example the British and US Pharmacopoeias, Remington's Pharmaceutical Sciences (Mack Publishing Co.), Martindale The Extra Pharmacopoeia (London, The Pharmaceutical Press) and Harry's Cosmeticology (Leonard Hill Books).

The invention also provides the use of an insulin sensitiser, such as Compound (I) and especially 2 to 12 mg thereof, for the manufacture of a medicament for the treatment of hyperglycaemia, especially fasting hyperglycaemia, wherein plasma glucose levels are in the range of from >126 mg/dl to 140 mg/dl.

In addition, the invention also provides the use of an insulin sensitiser, such as Compound (I) and especially 2 to 12 mg thereof, for the manufacture of a medicament for improving glycaemic control in conditions characterised by hyperglycaemia, especially fasting hypergiycaemia, the improvement being provided wherein plasma glucose levels are in the range of from >126 mg/dl to 140 mg/dl, thereby delaying or preventing further elevation of the hypergylcaemia.

In yet a further aspect, the invention provides the use of an insulin sensitiser, such as Compound (I) and especially 2 to 12 mg thereof, for the manufacture of a medicament for the prophylaxis of hyperglycaemia, especially fasting hyperglycaemia, wherein plasma glucose levels are >140 mg/dl.

The present invention also provides a pharmaceutical composition comprising an insulin sensitiser, such as Compound (I) and especially 2 to 12 mg thereof, and a pharmaceutically acceptable carrier therefor, for use in the treatment of hyperglycaemia, especially fasting hyperglycaemia, wherein plasma glucose levels are in the range of from >126 mg/dl to 140 mg/dl or for the improvement of glycaemic control in conditions characterised by fasting hyperglycaemia, the improvement being provided in the range of hyperglycaemia wherein plasma glucose levels are in the range of from >126 mg/dl to 140 mg/dl, thereby delaying or preventing further elevation of the hypergylcaemia or for the prophylaxis of hyperglycaemia, especially fasting hyperglycaemia, wherein plasma glucose levels are >140 mg/dl. No adverse toxicological effects are expected for the compositions or methods of the invention in the above mentioned dosage ranges.

1-12 (Cancelled).

13. A method for the treatment of hyperglycaemia wherein plasma glucose levels are in the range from >126 mg/dl to 140 mg/dl, which method comprises administering an effective non-toxic and pharmaceutically acceptable amount of 5-[4-[2-(N-methyl-N-(2-pyridyl)amino)ethoxy]benzyl]thiazolidine-2,4-dione, or a tautomer thereof, or a pharmaceutically acceptable salt or solvate thereof, to a mammal in need thereof.

14. A method according to claim 13, wherein the hyperglycaemia is fasting hyperglycaemia.

15. A method according to claim 13, comprising administering 2 to 12 mg of 5-[4-[2-(N-methyl-N-(2-pyridyl)amino)ethoxy]benzyl]thiazolidine-2,4-dione, or a tautomer thereof, per day.

16. A method according to claim 13, wherein the 5-[4-[2-(N-methyl-N-(2-pyridyl)amino)ethoxy]benzyl]thiazolidine-2,4-dione is in the form of a composition adapted for oral administration.

17. A method according to claim 16, wherein the composition is in unit dosage form.

18. A method according to claim 13, which comprises administering 2 mg of 5-[4-[2-(N-methyl-N-(2-pyridyl)amino)ethoxy]benzyl]thiazolidine-2,4-dione, or a tautomer thereof, per day.

19. A method according to claim 13, which comprises administering 4 mg of 5-[4-[2-(N-methyl-N-(2-pyridyl)amino)ethoxy]benzyl]thiazolidine-2,4-dione, or a tautomer thereof, per day.

20. A method according to claim 13, which comprises administering 8 mg of 5-[4-[2-(N-methyl-N-(2-pyridyl)amino)ethoxy]benzyl]thiazolidine-2,4-dione, or a tautomer thereof, per day.

21. A method according to claim 13, which comprises administering 5-[4-[2-(N-methyl-N-(2-pyridyl)amino)ethoxy]benzyl]thiazolidine-2,4-dione, or a tautomer thereof, in a pharmaceutically acceptable form.

22. A method according to claim 13, wherein said 5-[4-[2-(N-methyl-N-(2-pyridyl)amino)ethoxy]benzyl]thiazolidine-2,4-dione, or tautomer thereof is in the form of a pharmaceutically acceptable salt.

23. A method according to claim 22, wherein said pharmaceutically acceptable salt is a maleate salt.

24. A method according to claim 14, wherein the fasting plasma glucose level of said fasting hyperglycaemia is in the range of from >126 mg/dl to 140 mg/dl.

25. A method according to claim 24, further comprising treating hyperglycemia, wherein the plasma glucose level following an oral glucose tolerance test is <140 mg/dl.

26. A method according to claim 24, further comprising treating hyperglycemia, wherein the plasma glucose level following an oral glucose tolerance test is from 140 to <200 mg/dl.
